(19) United States
(12) Patent Application Publication
(10) Pub. No.: US 2012/0101160 A1
(43) Pub. Date: Apr. 26, 2012

(54) Use of chemical chaperones to treat glaucoma caused by misfolded or misprocessed proteins

(75) Inventors: Allan Shepard, Fort Worth, TX (US); Nasreen Jacobson, Fort Worth, TX (US); Abbot F. Clark, Arlington, TX (US)

(73) Assignee: Alcon, Inc.

(21) Appl. No.: 11/302,839

(22) Filed: Dec. 14, 2005

Related U.S. Application Data
(60) Provisional application No. 60/637,520, filed on Dec. 20, 2004.

(51) Int. Cl. A61K 31/192; A61P 27/06; A61K 31/131; A61K 31/16; A61K 31/205; A61K 31/10

(52) U.S. Cl. 514/556; 514/570; 514/708; 514/644; 514/616

(57) ABSTRACT

The present invention provides methods for treating glaucoma by administering compounds capable of stabilizing misprocessed or misfolded proteins that are responsible for the condition.

This application claims priority from the provisional application, U.S. Patent Application Ser. No. 60/637,520 filed Dec. 20, 2004.

### BACKGROUND OF THE INVENTION

1. Field of the Invention

The present invention is in the fields of diagnostics, and concerns methods and reagents for diagnosing and treating glaucoma and related disorders.

2. Description of the Related Art

“Glaucomas” are a group of debilitating eye diseases that are the leading cause of irreversible blindness in the United States and other developed nations. Primary Open Angle Glaucoma (“POAG”) is the most common form of glaucoma. The disease is characterized by the degeneration of the trabecular meshwork, leading to obstruction of the normal ability of aqueous humor to leave the eye without closure of the space (e.g., the “angle”) between the iris and cornea (Vaughan, D. et al., (1992)). A characteristic of such obstruction in this disease is an increased intraocular pressure (“IOP”), resulting in progressive visual loss and blindness if not treated appropriately and in a timely fashion. The disease is estimated to affect between 0.4% and 3.3% of all adults over 40 years old (Leske, M. C. et al. (1986); Bengtsson, B. (1989); Strong, N. P. (1992)). Moreover, the prevalence of the disease rises with age to over 6% of those 75 years or older (Strong, N. P., (1992)).

Glaucoma affects three separate tissues in the eye. The elevated IOP associated with POAG is due to morphological and biochemical changes in the trabecular meshwork (TM), a tissue located at the angle between the cornea and iris. Most of the nutritive aqueous humor exits the anterior segment of the eye through the TM. The progressive loss of TM cells and the build-up of extracellular debris in the TM of glaucomatous eyes leads to increased resistance to aqueous outflow (Lutjen-Drecoll and Rohen 1996; Rohen 1983; Rohen et al. 1993; Grierson and Calthorpe 1988), thereby raising IOP. Elevated IOP, as well as other factors such as ischemia, cause degenerative changes in the optic nerve head (ONH) leading to progressive “cupping” of the ONH (Varma and Minckler 1996; Hernandez and Gong 1996; Hernandez et al. 1990; Hernandez and Pena 1997; Morrison et al. 1990) and loss of retinal ganglion cells (Quigley et al. 2000; Quigley 1999; Quigley et al. 1995; Kerrigan et al. 1997) and axons. The detailed molecular mechanisms responsible for glaucomatous damage to the TM, ONH, and the retinal ganglion cells are unknown.

Disruption of normal aqueous outflow leading to elevated intraocular pressure (IOP) is integral to glaucoma pathophysiology. Current glaucoma therapy is directed to lowering IOP, a major risk factor for the development and progression of glaucoma. These therapies lower IOP by the use of suppressants of aqueous humor formation, agents that enhance uveoscleral outflow, trabeculoplasty, or trabeculaectomy. The current therapies do not directly address the pathological damage to the trabecular meshwork, which continues unabated.

Some forms of glaucoma are caused, at least part, by mutations in certain genes involved in ocular pathways. For example, within the past 8 years, over 15 different glaucoma genes have been mapped and 7 glaucoma genes identified. This includes six mapped genes (GLC1A-GLC1F) and two identified genes (MYOC and OPTN) for primary open angle glaucoma, two mapped genes (GLC3A-GLC3B) and one identified gene for congentical glaucoma (CY1B1), two mapped genes for pigmentary dispersion/pigmentary glaucoma, and a number of genes for developmental or syndromic forms of glaucoma (FOXC1, PITX2, LMX1B, PAX6).

In view of the importance of glaucoma, and the at least partial inadequacies of prior methods of treatment, it would be desirable to be able to identify compounds, and to have treatments, that will directly interfere with the pathogenic process, thereby protecting or rescuing patients from the damage caused by glaucoma.

### SUMMARY OF THE INVENTION

The present invention overcomes these and other drawbacks of the prior art by providing a method of treating glaucoma resulting from cellular misprocessed or misfolded proteins. The method of the invention comprises administering to a mammal in need thereof a therapeutically effective amount of a composition including at least one compound capable of stabilizing the misprocessed or misfolded protein such that the protein enters the normal processing pathway.

In another aspect, the present invention provides a method for treating glaucoma by targeting the Unfolded Protein Response (UPR) or Endoplasmic Reticulum-Associated Protein Degradation (ERAD) pathways. This method comprises administering to a mammal suffering from glaucoma resulting from cellular misprocessed or misfolded protein, a therapeutically effective amount of a composition containing a compound capable of stabilizing the misprocessed or misfolded protein such that the UPR or ERAD pathways are inactivated.

In preferred embodiments, the misprocessed or misfolded protein that is affected by the compositions of the invention is myocilin. Generally, the compound capable of stabilizing the misprocessed or misfolded protein in the methods of the invention may be 4-phenylbutyric acid, glycerol, betaine, dimethylsulfoxide, trimethylamine oxide and deoxyspergualin. The preferred concentration of the compound in the composition will be from about 0.01% to about 2%. The composition of the invention will typically be administered topically, intracamerally or via an implant.

It is contemplated that compounds useful in the methods of the present invention will in no way be limited to peptides or peptide mimetics. In fact, it may prove to be the case that the most useful pharmacological compounds will be non-peptidyl in nature and serve to stabilize the misprocessed or misfolded protein through a tight binding or other chemical interaction.

### BRIEF DESCRIPTION OF THE DRAWINGS

FIG. 1. Treatment with 10% glycerol of CHO cells expressing lacZ, wild-type myocilin, or mutant myocilin E and I indicate extracellular and intracellular myocilin. Molecular mass marker sizes (in kD) are indicated on the left. The relative migration of full-length and truncated (Q368X) myocilin is indicated on the right.

FIG. 2. Treatment with gentamycin of CHO cells expressing lacZ, wild-type myocilin, or mutant myocilin. E and I indicate extracellular and intracellular myocilin. Molecular mass marker sizes (in kD) are indicated on the left. The relative migration of full-length and truncated (Q368X) myocilin is indicated on the right.

### DETAILED DESCRIPTION PREFERRED EMBODIMENTS

Glaucoma is a heterogeneous group of optic neuropathies that share certain clinical features. The loss of vision in glaucoma is due to the selective death of retinal ganglion cells in the neural retina that is clinically diagnosed by characteristic changes in the visual field, nerve fiber layer defects, and a progressive cupping of the ONH. One of the main risk factors for the development of glaucoma is the presence of ocular hypertension (elevated intraocular pressure, IOP). IOP also appears to be involved in the pathogenesis of normal tension glaucoma where patients have what is often considered to be normal IOP. The elevated IOP associated with glaucoma is due to elevated aqueous humor outflow resistance in the trabecular meshwork (TM), a small specialized tissue located in the iris-corneal angle of the ocular anterior chamber. Glaucomatous changes to the TM include a loss in TM cells and the deposition and accumulation of extracellular debris including plaque-like material. In addition, there also are changes that occur in the glaucomatous optic nerve head. In glaucomatous eyes, there are morphological and mobility changes in ONH glial cells. In response to elevated IOP and/or transient ischemic insults, there is a change in the composition of the ONH extracellular matrix and alterations in the glial cell and retinal ganglion cell axon morphologies.

A number of diseases are known to be caused by misfolded or misprocessed proteins. For example, in α1-AT deficiency, a misfolded but functionally active mutant α1-ATZ (α1-ATZ) molecule is retained in the endoplasmic reticulum of liver cells rather than secreted into the blood and body fluids. Emphysema is thought to be caused by the lack of circulating α1-AT to inhibit neutrophil elastase in the lung. (Burrows et al. 2000; Novoradovskaya et al. 1998). Other diseases that appear to be caused by misfolded proteins include kuru, Creutzfeldt-Jakob disease (CJD), Gerstmann-Sträussler-Scheinker syndrome (GSS) (Tatzelt et al. 1996), Fabry disease (Fan et al. 1999), non-X-linked nephrogenic diabetes insipidis (NDI) (Tamarappoo and Verkman 1998), and cystic fibrosis (CF) (Sato et al. 1996).

Chemical chaperones are compounds that stabilize proteins in their native conformation or that facilitate proper protein folding (Brown et al. 1997; Smith et al. 1998). Chemical chaperones have been shown to correct the mutant phenotype of the ΔF508 cystic fibrosis transmembrane conductance regulator protein (CFTR), thereby allowing it to be transported to the plasma membrane where it functions, similar to the wild-type protein, in mediating chloride transport. Brown et al. (1996) showed that compounds such as glycerol, trimethylamine N-oxide and deuterated water restored the ability of the mutant cells to exhibit forskolin-dependent chloride transport, similar to that observed for the cells expressing the wild-type CFTR protein.

The use of chemical chaperones to treat or rescue patients from the damage caused by glaucoma has not been taught or suggested prior to the present invention. The fundamental principle behind using chemical chaperones in the treatment of glaucoma is that diesease-causing misfolded or misprocessed proteins may be refolded or allowed to fold correctly in cells treated with chaperones thereby alleviating the disease process. Misfolded proteins are known to activate the Unfolded Protein Response (UPR) or Endoplasmic Reticulum-Associated Protein Degradation (ERAD) pathways (Travers et al. 2000). Chemical chaperones have been shown to alleviate this process and generate properly folded and even functional protein (Brown et al. 1996; Rubenstein and Zeitlin 1998). Simple relief of the UPR/ERAD processes may be sufficient to correct or prevent disease. For certain glaucoma-causing genes, such as myocilin, the trabecular meshwork (TM) and other tissues of the eye may be less efficient than non-ocular tissues at processing these mutant proteins.

The therapeutic agent for the treatment of glaucoma can be: a peptide or protein, a peptide mimetic, an oligonucleotide or derivatized oligonucleotide, or small drug-like molecule, all which affect one or more aspects of the ocular UPR/ERAD pathways. Preferred therapeutic agents are those that are able to stabilize a misprocessed or misfolded protein such that it is able to substantially function as the wild-type protein functions in its normal environment.

The agents of this invention, can be incorporated into various types of ophthalmic formulations for delivery to the eye (e.g., topically, intracamerally, juxtasclerally, or via a cannula or an implant). The agents are preferably incorporated into topical ophthalmic formulations for delivery to the eye. The agents may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving an agent in a physiologically acceptable isotonic aqueous buffer. Further, the ophthalmic solution may include an ophthalmologically acceptable surfactant to assist in dissolving the agent. Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the agent in a hydrophilic base prepared from the combination of, for example, carbopol-974, or the like, according to the published formulations for analogous ophthalmic preparations; preservatives and tonicity agents can be incorporated.

The agents are preferably formulated as topical ophthalmic suspensions or solutions, with a pH of about 4 to 8. The establishment of a specific dosage regimen for each individual is left to the discretion of the clinicians. The agents will normally be contained in these formulations in an amount 0.01% to 5% by weight, but preferably in an amount of 0.05% to 2% and most preferably in an amount 0.1 to 1.0% by weight. The dosage form may be a solution, suspension microemulsion. Thus, for topical presentation 1 to 2 drops of these formulations would be delivered to the surface of the eye 1 to 4 times per day according to the discretion of a skilled clinician.

The agents can also be used in combination with other agents for treating glaucoma, such as, but not limited to, β-blockers, prostaglandin analogs, carbonic anhydrase inhibitors, α₂ agonists, miotics, and neuroprotectants.

The agent may be delivered directly to the eye (for example: topical ocular drops or ointments; slow release devices in the cul-de-sac or implanted adjacent to the sclera or within the eye; periocular, conjunctival, sub-Tenons, intracameral or intravitreal injections) or parenterally (for example: orally; intravenous, subcutaneous or intramuscular injections; dermal delivery; etc.) using techniques well known by those skilled in the art. The following is an example of a possible formulation embodied by this invention.

| Topical ocular formulation | wt. % |
|---|---|
| Compound that stabilizes misfolded | 0.01-2 |
| or misprocessed protein | |
| HPMC | 0.5 |
| Sodium chloride | 0.8 |
| BAC | 0.01% |
| EDTA | 0.01 |
| NaOH/HCl | qs pH 7.4 |
| Purified water | qs 100 mL |

It is further contemplated that the compounds of the invention could be formulated in intraocular insert devices.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Wild-type myocilin is normally expressed and secreted from the human TM, whereas mutant disease-causing myocilin is retained intracellularly (Jacobson, Andrews et al. 2001). Overcoming the intracellular retention of mutant myocilin by the treatment with chemical chaperones may prove therapeutic and as such, was used as a model system. We tested several compounds for their ability to effect secretion of mutant myocilin in cultured CHO cells that were engineered for their ability to express, but not secrete, mutant (Y437H or Q368X) myocilin. Plasmids expressing wild-type or mutant myocilin were generated as described (Jacobson, Andrews et al. 2001).

CHO stable transfectants were grown in F12K media containing 10% FBS supplemented with penicillin-streptomycin-glutamine. Stable transfectants were generated by transfection with lipofectamine plus (Invitrogen) followed by limiting dilution plating and selection in G-418 (600 ug/ml). CHO cells were incubated with serum-free media 24 h post-transfection and media or cell lysates were collected after overnight incubation with the chemical chaperone. Equivalent cell lysate protein amounts (5 ug) were loaded for SDS-PAGE analysis. Cell media was not concentrated and was loaded at 16 ul per lane for SDS-PAGE/Western blot analysis.

Cells were rinsed with PBS and solubilized in a commercial mammalian extraction buffer (M-Per™; Pierce, Rockford, Ill.) supplemented with a protease inhibitor cocktail (Complete, EDTA-free; Boehringer Mannheim, Indianapolis, Ind.) followed by centrifugation at 12,000×g for 5 min. Protein concentration of the supernatant was determined with Coomasie Plus Protein Assay Reagent (Pierce). Cell extracts were stored at −20° C.

Cell media or extracts were analyzed using pre-cast NuPAGE polyacrylamide gels and the Novex gel electrophoresis system (Novex, San Diego, Calif.). Proteins were electroblotted to Hybond-P PVDF membranes (Amersham Pharmacia Biotech, Piscataway, N.J.), blocked with gelatin, and probed with affinity purified rabbit anti-MYOC antibody 129 (generated to myocilin peptide 156-171) (Jacobson, Andrews et al. 2001) and an anti-rabbit IgG secondary antibody (Amersham). Immunoreactivity was detected with the ECL Plus detection system (Amersham). Blots were exposed to BioMax MR film (Eastman Kodak, Rochester, N.Y.) and scanned with a Hewlett-Packard ScanJet ADF scanner (Hewlett Packard, Boise, Id.) for figure presentation.

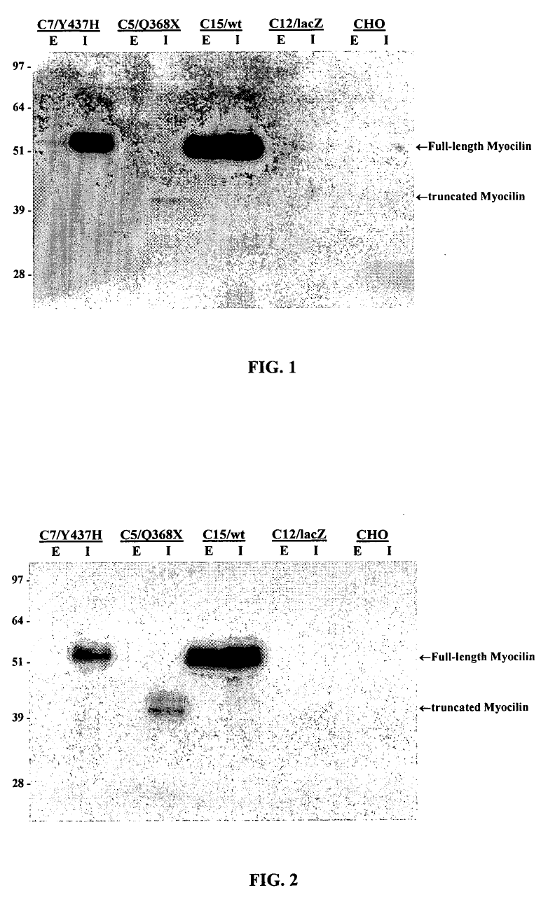

Glycerol is shown as an example of a chemical chaperone that is able to effect full-length myocilin secretion in the Q368X (E and I lanes) and Y437H (E lane) mutant cell lines (FIG. 1). Control untransfected and lacZ stably transfected CHO cells were negative for myocilin expression. Gentamycin is shown as an example of a chemical chaperone that did not effect secretion of mutant myocilin under our conditions (FIG. 2) and is useful as a comparison to the results of FIG. 1.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents

Books

Other Publications
Brown C R, Hong-Brown L Q, Biwersi J, Verkman A S, Welch W J, “Chemical chaperones correct the mutant phenotype of the ΔF508 cystic fibrosis transmembrane conductance regulator protein,” Cell Stress Chaperones 1(2):117-125 (1996).
Brown C R, Hong-Brown L Q, Welch W J, “Correcting Temperature-sensitive Protein Folding Defects,” J. Clin. Invest. 99(6):1432-1444 (1997).
Burrows J A J, Willis L K, Perlmutter D H, “Chemical chaperones mediate increased secretion of mutant α1-antitrypsin (α1-AT) Z: A potential pharmacological strategy for prevention of liver injury and emphysema in α1-AT deficiency,” PNAS 97(4):1796-1801 (2000).
Diamant S, Eliahu N, Rosenthal D, Goloubinoff P, “Chemical Chaperones Regulate Molecular Chaperones in vitro and In Cells Under Combined Salt and Heat-stresses,” JBC Papers in Press, Manuscript M103081200 (2001).
Fan J Q, Ishi S, Asano N, Suzuki Y, “Accelerated transport and maturation of lysosomal α-galactosidase A in Fabry lymphoblasts by and enzyme inhibitor,” Nature Medicine 5(1):112-115 (1999).
Jacobson, N., Andrews, M., Shepard, A. R., Nishimura, D., Searby, C., Fingert, J. H., Hageman, G., Mullins, R., Davidson, B. L. Kwon, Y. H., Alward, W. L., Stone, E. M., Clark, A. F., Sheffield, V. C., “Non-secretion of mutant proteins of the glaucoma gene myocilin in cultured trabecular meshwork cells and in aqueous humor,” Hum. Mol. Gen. 10(2):117-125 (2001).
Jiang C, Fang S L, Xiao Y F, O'Connor S P, Nadler S G, Lee D W, Jefferson D M, Kaplan J M, Smith A E, Cheng S H, “Partial restoration of cAMP-stimulated CFTR chloride channel activity in ΔF508 cells by deoxyspergualin,” Am. J. Physiol. 275:C171-178 (1998).
Novoradovskaya N, Lee J, Yu, Z X, Ferrans V J, Brantly M, “Inhibition of Intracellular Degradation Increases Secretion of a Mutant Form of α1-Antitrypsin Associated with Profound Deficiency,” J. Clin. Invest. 101(12):2693-2701 (1998).
Rubenstein R C, Egan M E, Zeitlin P L, “In Vitro Pharmacologic Restoration of CFTR-mediated Chloride Transport with Sodium 4-Phenylbutyrate in Cystic Fibrosis Epithelial Cells Containing ΔF508-CFTR,” J. Clin. Invest. 100(10):2457-2465 (1997).
Rubenstein R C, Zeitlin P L, “A Pilot Clinical Trial of Oral Sodium 4-Phenylbutyrate (Buphenyl) in ΔF508-Homozygous Cystic Fibrosis Patients,” Am. J. Respir. Crit. Care Med. 157:484-490(1998).
Rubenstein R C, Zeitlin P L, “Sodium 4-phenylbutyrate downregulates Hsc70: implications for intracellular trafficking of ΔF508-CFTR,” Am. J. Physiol. Cell Physiol. 278:C259-C267 (2000).
Sato S, Ward C L, Krouse M E, Wine J J, Kopito R R, “Glycerol Reverses the Misfolding Phenotype of the Most Common Cystic Fibrosis Mutation,” J. Biol. Chem. 271(2):635-638 (1996).
Smith D F, Whitesell L, Katsanis E, “Molecular Chaperones: Biology and Prospects for Pharmacological Intervention,” Pharm. Reviews 50(4):493-513 (1998).
Tamarappoo B K, Verkman A S, “Defective Aquaporin-2 Trafficking in Nephrogenic Diabetes Insipidus and Correction by Chemical Chaperones,” J. Clin. Invest. 101(10):2257-2267 (1998).
Tatzelt J, Prusiner S B, Welch W J, “Chemical chaperones interfere with the formation of scrapie prion protein,” Embo J 15:6363-6373 (1996).

1. A method of treating glaucoma resulting from cellular misprocessed or misfolded proteins, said method comprising administering to the eye of a mammal in need thereof a therapeutically effective amount of a composition comprising at least one compound capable of stabilizing said misprocessed or misfolded protein such that it enters the normal processing pathway, wherein said misprocessed or misfolded protein is myocilin and wherein said compound is selected from the group consisting of 4-phenylbutyric acid, betaine, dimethylsulfoxide, trimethylamine oxide and deoxyspergualin.

2. (canceled)

3. (canceled)

4. The method of claim 1, wherein said composition is administered via a route of administration selected from the group consisting of topical ocular administration, intracamerally, and via an ocular implant.

5. The method of claim 1, wherein the total concentration of said compound in said composition is from 0.01% to 2%.

6. A method for treating glaucoma by targeting the Unfolded Protein Response (UPR) or Endoplasmic Reticulum-Associated Protein Degradation (ERAD) pathways, said method comprising administering to a mammal suffering from glaucoma resulting from cellular misprocessed or misfolded protein a therapeutically effective amount of a composition comprising a compound capable of stabilizing said misprocessed or misfolded protein such that the UPR or ERAD pathways are inactivated, wherein said misprocessed or misfolded protein is myocilin and wherein the compound is selected from the group consisting of 4-phenylbutyric acid, betaine, dimethylsulfoxide, trimethylamine oxide, and deoxyspergualin.

7. (canceled)

8. (canceled)
